(19) United States
(12) Patent Application Publication
(10) Pub. No.: US 2024/0209077 A1
(43) Pub. Date: Jun. 27, 2024

(54) POLYCLONAL ANTIBODY TARGETING SODIUM ION CHANNEL, PHARMACEUTICAL COMPOSITION COMPRISING POLYCLONAL ANTIBODY TARGETING SODIUM ION CHANNEL, AND METHOD FOR TREATING AND/OR PREVENTING CARDIAC ARRHYTHMIA BY USING POLYCLONAL ANTIBODY TARGETING SODIUM ION CHANNEL

(71) Applicant: National Taiwan University, Taipei City (TW)

(72) Inventor: Chia-Ti Tsai, Taipei City (TW)

(21) Appl. No.: 18/121,285

(22) Filed: Mar. 14, 2023

(30) Foreign Application Priority Data
Dec. 26, 2022 (TW) 111149872

(51) Int. Cl. C07K 16/28; A61K 9/00; A61P 9/06

(52) U.S. Cl. CPC C07K 16/28; A61K 9/0019; A61P 9/06

(57) ABSTRACT

The present disclosure provides a polyclonal antibody targeting sodium ion channel, which binds to a sodium Nav1.5 channel. The present disclosure also provides a pharmaceutical composition including the polyclonal antibody targeting sodium ion channel, and a method for treating and/or preventing cardiac arrhythmia by using the polyclonal antibody targeting sodium ion channel.

### CROSS-REFERENCE TO RELATED APPLICATION

This application claims priority of Taiwan patent application No. 111149872, filed on Dec. 26, 2022, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 111F0297-IE_Sequence_listing. The XML file is 3000 bytes; was created on Dec. 26, 2022.

### BACKGROUND OF THE INVENTION

### 1. Field of the Invention

The present invention relates to a polyclonal antibody targeting sodium ion channel, a pharmaceutical composition comprising the polyclonal antibody targeting sodium ion channel, and a method for treating and/or preventing cardiac arrhythmia by using the polyclonal antibody targeting sodium ion channel.

### 2. The Prior Art

Heart disease has always been one of the leading causes of death in countries all over the world. The reason is that as long as myocardial cells are damaged and dead, the health damage caused to the individual is difficult to recover. Atrial fibrillation (AF) is the most common sustained arrhythmia and is a major public health problem in the world. AF is associated with substantial mortality, morbidity, and health care costs. AF can result in myocardial ischemia and infarction and exacerbation of heart failure. The most disastrous complication of AF is thromboembolism, especially stroke. The therapy of AF and its complications is responsible for a substantial proportion of cost of health care system per year all over the world. There are several types of AF. In patients with paroxysmal AF (PAF), the AF rhythm may terminate spontaneously, but the patients may suffer from repeated paroxysmal attacks of AF. In patients with persistent AF, the AF rhythm does not self terminate and electrical cardioversion or acute use of anti-arrhythmic drugs (AADs) are needed to convert the AF rhythm back to normal sinus rhythm. In patients with permanent or chronic AF, the AF rhythm is too long-standing and chronic, and conversion to sinus rhythm is not intended (too difficult to maintain normal sinus rhythm). AADs are used to convert AF to sinus rhythm or maintain sinus rhythm for patients with PAF or persistent AF, and are called rhythm control agents. For patients with permanent AF, conversion to sinus rhythm is not intended, and only ventricular rate is kept within the normal range to treat symptoms of palpitation by drugs that inhibit atrioventricular node conduction, which are called rate control agents.

Although AF is an important cardiovascular disease, the medical treatment to maintain normal sinus rhythm is far from satisfactory. Class I (sodium channel blocker) and class III AADs (potassium channel blocker) are the standard treatment for normal sinus rhythm control. However, use of these AADs is associated with potential serious side effects. The most commonly used class III AAD is amiodarone, use of which is often associated with thyroid dysfunction, pulmonary fibrosis, hepatic dysfunction and aggravation of heart failure. The most commonly used class I AAD is propafenone, use of which is often associated with conduction block, bradycardia, and predisposition to ventricular arrhythmia and sudden cardiac death. Regarding the drugs for rate control, beta blocker and verapamil are associated with the side effect of myocardial suppression and may predispose to acute congestive heart failure. Digoxin can cause digitalis intoxication and is also proarrhythmic. Recently, ivabradine has also been proposed as an alternative drug for rate control. However, its effect has not been well proved. Therefore, identification of a safer drug for either rhythm or rate control in AF patients is mandatory.

In order to solve the above-mentioned problems, those skilled in the art urgently need to develop a novel and more effective method with fewer side effects for treating and/or preventing cardiac arrhythmia for the benefit of a large group of people in need thereof.

### SUMMARY OF THE INVENTION

A primary objective of the present invention is to provide a polyclonal antibody targeting sodium ion channel, which binds to a sodium Nav1.5 channel, wherein the sodium Nav1.5 channel has a Nav1.5 pore-forming region, and the polyclonal antibody targeting sodium ion channel binds to the Nav1.5 pore-forming region.

Another objective of the present invention is to provide a pharmaceutical composition, comprising the aforementioned polyclonal antibody targeting sodium ion channel, and a pharmaceutically acceptable carrier.

Another objective of the present invention is to provide a method for treating and/or preventing cardiac arrhythmia, comprising administering to a subject in need thereof the pharmaceutical composition comprising an effective amount of the aforementioned polyclonal antibody targeting sodium ion channel.

According to an embodiment of the present invention, the sodium Nav1.5 channel is a cardiac sodium Nav1.5 channel.

According to an embodiment of the present invention, the polyclonal antibody targeting sodium ion channel blocks sodium ions from passing through the cardiac sodium Nav1.5 channel.

According to an embodiment of the present invention, the Nav1.5 pore-forming region comprises an amino acid sequence of SEQ ID NO: 1.

According to an embodiment of the present invention, the cardiac arrhythmia is atrial arrhythmia or atrial fibrillation.

According to an embodiment of the present invention, the pharmaceutical composition is in a dosage form for parenteral administration.

According to an embodiment of the present invention, the arrhythmia is treated and/or prevented by blocking sodium ions from passing through the sodium Nav1.5 channel by the polyclonal antibody targeting sodium ion channel.

In summary, the polyclonal antibody targeting sodium ion channel of the present invention has the effect on blocking sodium ions from passing through the cardiac sodium Nav1.5 channel to treat and/or prevent cardiac arrhythmia.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the three dimensional structure of cardiac sodium channel. The cardiac sodium channel is composed of 4 same Nav1.5 subunits encoded by the SCN5A gene. The dark part is the pore-forming region and is the target of our generated polyclonal antibodies. The left panel shows the lateral view of cardiac sodium channel and arrow indicates the direction of sodium (Na⁺) influx from the extracellular space into the cytosol through the cardiac sodium channel. The right panel shows the enface view looking at the pore-forming region of the cardiac sodium channel.

FIGS. 2A and 2B show the Western blot results of the polyclonal antibody targeting sodium ion channel (hereinafter referred to as “Nav1.5 polyclonal antibody”). FIG. 2A, upper panel, total protein was extracted from HL-1 cardiomyocytes and western blot was performed using our generated Nav1.5 polyclonal antibody from Example 1. A band at around 185 kDA was the postulated band of Nav1.5. Loading control using glyceraldehyde-3-phosphate dehydrogenase (GAPDH) antibody is also shown (FIG. 2A, lower panel). FIG. 2B, Western blot using the synthesized peptide from 2.5 μg to 30 μg and Nav1.5 polyclonal antibody is shown.

FIG. 3 shows the electrocardiogram of a rat receiving Nav1.5 polyclonal antibody injection. Atrioventricular conduction block was noted with a 4 to 1 atrioventricular conduction. The arrows indicate the P waves, which mean that in a complete cardiac potential change, the first deviation that occurs is called the P wave caused by atrial contraction.

FIG. 4 shows the electrocardiograms of rats receiving Nav1.5 polyclonal antibody injection. Rats received tracheostomy and mechanical ventilation. Sternotomy was performed and the intact heart was exposed. A pacing lead was placed on the right atrium for tachypacing at 100 Hz. Atrial arrhythmia was induced by tachypacing and transient asphyxia by stop of mechanical ventilation. FIG. 4A, a representative control electrocardiogram before arrhythmia induction. FIG. 4B, a representative induced atrial arrhythmia with variable atrioventricular conduction. The arrows indicate P waves. We induced atrial arrhythmia by right atrial tachypacing at 100 Hz and transient asphyxia by termination of ventilation. FIG. 4C, a representative electrocardiogram with pre-treatment of Nav1.5 polyclonal antibody and then atrial arrhythmia was induced. Atrial arrhythmia was not inducible and sinus rhythm was noted after arrhythmia induction. FIG. 4D, another representative electrocardiogram with pre-treatment of Nav1.5 polyclonal antibody and then atrial arrhythmia was induced. Atrial arrhythmia was not inducible and sinus rhythm with atrioventricular conduction block was noted after arrhythmia induction.

FIGS. 5A and 5B show bar graphs of mean P-P intervals and R-R intervals in electrocardiogram. FIG. 5A, mean P-P intervals of control rats (CTL), those with atrial arrhythmia induction (turn off the respirator) and those with pre-treatment of Nav1.5 polyclonal antibody and atrial arrhythmia induction (turn off the respirator+Nav1.5 Ab). The mean P-P interval of atrial arrhythmia was shorter (faster rate) than that of normal sinus rhythm. The mean P-P interval of atrial arrhythmia was also shorter than that of sinus rhythm after pre-treatment of Nav1.5 polyclonal antibody and atrial arrhythmia induction. CTL means control, and Nav1.5 Ab means administration of the Nav1.5 polyclonal antibody. FIG. 5B, mean R-R intervals of control rats, those with atrial arrhythmia induction and those with pre-treatment of Nav1.5 polyclonal antibody and atrial arrhythmia induction. CTL means control, and Nav1.5 Ab means the Nav1.5 polyclonal antibody. The mean R-R interval of atrial arrhythmia was shorter (faster rate) than that of normal sinus rhythm. The mean R-R interval of atrial arrhythmia was also shorter than that of sinus rhythm after pre-treatment of Nav1.5 polyclonal antibody and atrial arrhythmia induction.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of +20%, preferably within +10%, and most preferably within +5%.

As used herein, the term “treating” or “treatment” refers to alleviating, reducing, ameliorating, relieving or controlling one or more clinical signs of a disease or disorder, and lowering, stopping, or reversing the progression of severity regarding the condition or symptom being treated.

As used herein, the term “preventing” or “prevention” means preventing or delaying symptoms of disease onset when a drug or therapy is used to an individual who does not have symptoms of disease onset but is at high risk of disease onset.

According to the present invention, the pharmaceutical composition can be manufactured to a dosage form suitable for parenteral administration, using techniques well known to those skilled in the art, including, but not limited to, injection (e.g., sterile aqueous solution or dispersion), sterile powder, tablet, troche, lozenge, pill, capsule, dispersible powder or granule, solution, suspension, emulsion, syrup, elixir, slurry, and the like.

The pharmaceutical composition according to the present invention may be administered by a parenteral route selected from the group consisting of: intraperitoneal injection, subcutaneous injection, intraepidermal injection, intradermal injection, intramuscular injection, intravenous injection, and intralesional injection.

According to the present invention, the pharmaceutical composition may further comprise a pharmaceutically acceptable carrier which is widely used in pharmaceutically manufacturing techniques. For example, the pharmaceutically acceptable carrier can comprise one or more reagents selected from the group consisting of solvent, emulsifier, suspending agent, decomposer, binding agent, excipient, stabilizing agent, chelating agent, diluent, gelling agent, preservative, lubricant, absorption delaying agent, liposome, and the like. The selection and quantity of these reagents fall within the scope of the professional literacy and routine techniques of those skilled in the art.

According to the present invention, the pharmaceutically acceptable carrier comprises a solvent selected from the group consisting of water, normal saline, phosphate buffered saline (PBS), sugar-containing solution, aqueous solution containing alcohol, and combinations thereof.

### Example 1

### Rabbit Immunization and Polyclonal Antibody Targeting Sodium Ion Channel Preparation

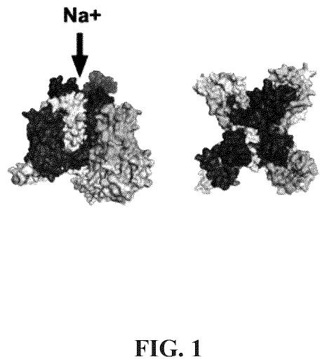

The peptide antigen of KNYSELRHRISDSG (SEQ ID NO: 1) that localizes in the pore-forming unit of Nav1.5 was synthesized (GenScript, Singapore) (hereinafter referred to as “synthetic peptide”) and conjugated it with Keyhole Limpet Hemocyanin (KLH) as immunogen (GenScript, Singapore). The location of the sequence can be seen from the lower right of FIG. 1. PolyExpress™ immunization strategy was applied to this project and antigen affinity purification (GenScript, Singapore). The targeted sequence of Nav1.5 at the pore-forming region is shown in FIG. 1.

FIG. 1 shows the three dimensional structure of cardiac sodium channel. The cardiac sodium channel is composed of 4 same Nav1.5 subunits encoded by the SCN5A gene. The red part is the pore-forming region and is the target of our generated polyclonal antibodies. The left panel shows the lateral view of cardiac sodium channel and arrow indicates the direction of sodium (Na⁺) influx from the extracellular space into the cytosol through the cardiac sodium channel. The right panel shows the enface view looking at the pore-forming region of the cardiac sodium channel.

The polyclonal antibody targeting sodium ion channel was generated by immunizing New Zealand White Rabbits with the synthesized peptide (SEQ ID NO: 1) per subcutaneous injection on DO, D14, and D21. The primary inoculation was with Freund's complete adjuvant (Sigma-aldrich, USA). The serum was pooled one week later after the last immunity. Blood was allowed to clot at 4° C., and the serum was removed from the top of the mixture after centrifugation. Immunoglobulin was purified by affinity chromatography (protein A sephrose) and freeze dried. The polyclonal antibody targeting sodium ion channel (hereinafter referred to as “Nav1.5 polyclonal antibody”) was kept frozen at −80° C. until used. The final antibody concentration was 0.512 mg/ml.

### Example 2

### Binding of Generated Nav1.5 Polyclonal Antibody to Cardiac Sodium Channels

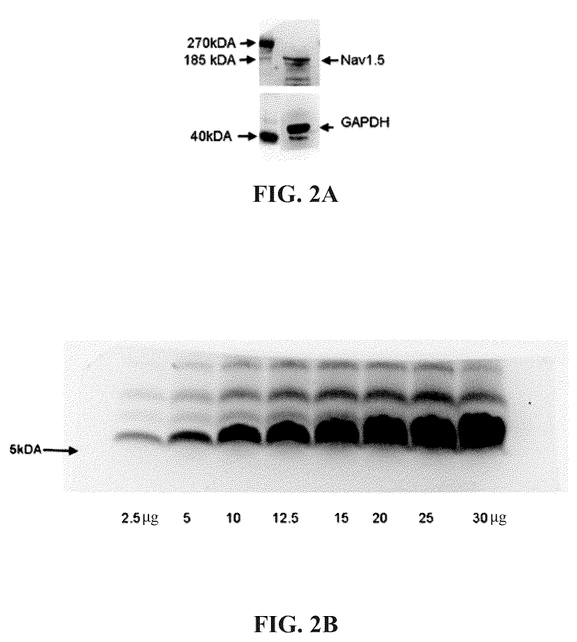

We first tested whether our generated Nav1.5 polyclonal antibodies in Example 1 bound to cardiac sodium channels. Total proteins were extracted from the HL-1 cardiomyocytes (Millipore) and Western blot using our generated Nav1.5 polyclonal antibodies as the primary antibody was performed. Culture of HL-1 cardiomyocytes is as follows. HL-1 myocytes were cultured in Claycomb medium. All of the experiments were conducted in a standardized fashion when the cells were in the confluent phase. The cells were collected for studies in the morning of the 4th day after subculture (one day of seeding and one day of serum-free growth). The cells were then lysed and total proteins were extracted. The procedure of Western blot analysis is as follows. The protein samples were subjected to sodium dodecyl sulfate-polyacrylamide gel electrophoresis and immuno-blotted with primary Nav1.5 polyclonal antibodies and then horseradish peroxidase-conjugated secondary antibody (Abcam). The proteins were visualized by enhanced chemiluminescence (Amersham). The result is shown in FIGS. 2A and 2B.

FIGS. 2A and 2B show the Western blot results of the polyclonal antibody targeting sodium ion channel (hereinafter referred to as “Nav1.5 polyclonal antibody”). FIG. 2A, upper panel, total protein was extracted from HL-1 cardiomyocytes and western blot was performed using our generated Nav1.5 polyclonal antibody from Example 1. A band at around 185 kDA was the postulated band of Nav1.5. Loading control using glyceraldehyde-3-phosphate dehydrogenase (GAPDH) antibody is also shown (FIG. 2A, lower panel). FIG. 2B, Western blot using the synthesized peptide (5 kDA) from 2.5 μg to 30 μg and Nav1.5 polyclonal antibody is shown.

We found that Nav1.5 polyclonal antibody of the present invention bound to sodium channel of mouse HL-1 cardiomyocytes (see the band in FIG. 2A). Then we used our synthesized peptides as the antigens and performed Western blot again. We also showed that Nav1.5 polyclonal antibody of the present invention bound to our synthesized peptide with a high affinity and dose dependent (see FIG. 2B). In addition, Table 1 also found that the polyclonal antibody diluted to 512000 times still has enzyme-linked immunosorbent assay (ELISA) activity.

We also confirmed this by the ELISA method. The procedure of ELISA is as follows. The wells were coated with our synthesized peptide. The wells were washed with phosphate buffered saline (PBS) three times before addition of Nav1.5 polyclonal antibodies. The polyclonal antibodies were serially diluted from 1:1000 to 1:512000 and incubated overnight at 4° C. The Nav1.5 polyclonal antibodies were then removed and the wells were washed with PBST (PBS containing 0.05% Tween-20) three times. The horseradish peroxidase (HRP) conjugated secondary antibody (1:5,000) was added to the wells and incubated for 30 min. Finally, add 100 μl of 3,3′,5,5′-tetramethylbenzidine (TMB) solution to each well and read absorbance at 450 nm. Each experiment was performed in triplicate. ELISA results of pre-immune serum and purified antibodies are shown in Table 1, representing that polyclonal antibodies can bind to the synthetic peptide.

TABLE 1

| | dilution | anti-Nav1.5 antibody |
|---|---|---|
| Pre-immune serum | 1:1000 | 0.069 |
| 1 | 1:1000 | 2.72 |
| 2 | 1:2000 | 2.634 |
| 3 | 1:4000 | 2.582 |
| 4 | 1:8000 | 2.569 |
| 5 | 1:16000 | 2.416 |
| 6 | 1:32000 | 2.296 |
| 7 | 1:64000 | 2.056 |
| 8 | 1:128000 | 1.723 |
| 9 | 1:256000 | 1.292 |
| 10 | 1:512000 | 0.914 |
| 11 | blank | 0.063 |
| 12 | blank | 0.063 |

### Example 3

### Effect of Nav1.5 Polyclonal Antibody on Normal Rat Electrocardiogram

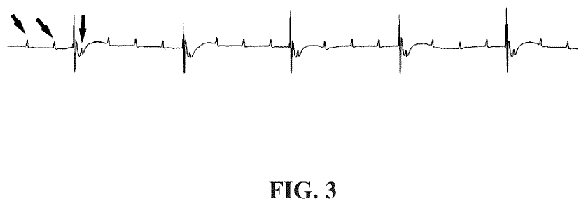

We first tested whether our Nav1.5 polyclonal antibodies affected cardiac electrophysiology in rats. We recorded electrocardiogram from a representative normal rat. The procedure is as follows. Wistar rats (300-350 g) received intraperitoneal injection of zoletil (20 mg/kg) prior to the electrophysiological studies. The ECG leads were fixed to the four limbs of the animals. The anesthetized mice were endotracheally intubated with a polyethylene tubing through an incision of the trachea and then mechanically ventilated (SAR-830 Small Animal Ventilator, CWE Inc., Ardmore, PA, USA). The chest was opened through sternal incision. Following pericardiotomy, a concentric bipolar stimulating electrode (SNE-100x 50 mm; RhodesMedical Instruments Inc. Summerland, CA, USA) was fixed to a micro-manipulator (Narishige, Japan) and attached to the right atrium for atrial tachypacing. Induction of atrial arrhythmia was performed by atrial tachypacing at 100 Hz and concomitant short-term asphyxia. All the electrophysiological data were recorded by the IX-214 Data Recorder (iWorx Systems, Inc., Dover, NH, USA) for off-line analysis. At the end of the study, the animals were euthanized by cervical dislocation. The experimental protocol conforms to the Guide for the Care and Use of Laboratory Animals (NIH Publication No. 85-23, revised 1996) and was approved by the Institutional Animal Care and Use Committee of the National Taiwan University College of Medicine. The result is shown in FIG. 3.

FIG. 3 shows the electrocardiogram of a rat receiving Nav1.5 polyclonal antibody injection. Atrioventricular conduction block was noted with a 4 to 1 atrioventricular conduction. The arrows indicate the P waves. We found that systemic administration of Nav1.5 (5.12 μg) caused significant atrioventricular (AV) conduction block in vivo, with little effect on heart rate and QRS duration, representing that the antibody binds to the cardiac Nav1.5 channel and inhibits the function of the channel, resulting in the therapeutic effect of atrioventricular conduction block (the atrioventricular node tissue contains Nav1.5 sodium channel).

### Example 4

### Effect of Nav1.5 Polyclonal Antibody on Prevention of Atrial Arrhythmia

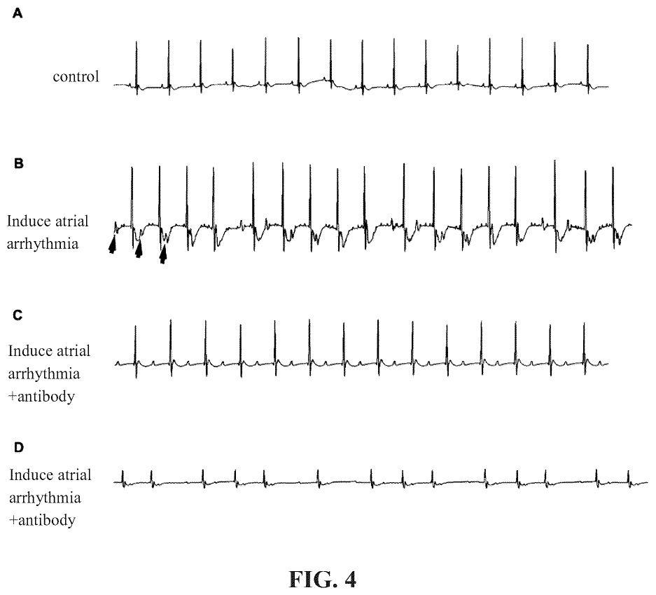

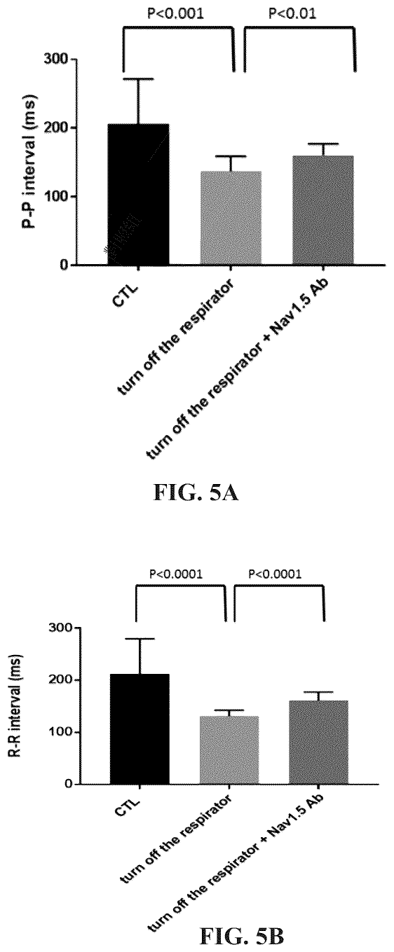

We then tested whether the Nav1.5 polyclonal antibodies could prevent atrial arrhythmia or AF. The results are shown in FIGS. 4-5B.

FIG. 4 shows the electrocardiograms of rats receiving Nav1.5 polyclonal antibody injection. Rats received tracheostomy and mechanical ventilation. Sternotomy was performed and the intact heart was exposed. A pacing lead was placed on the right atrium for tachypacing at 100 Hz. Atrial arrhythmia was induced by tachypacing and transient asphyxia by stop of mechanical ventilation. FIG. 4A, a representative control electrocardiogram before arrhythmia induction. FIG. 4B, a representative induced atrial arrhythmia with variable atrioventricular conduction. The arrows indicate P waves. We induced atrial arrhythmia by right atrial tachypacing at 100 Hz and transient asphyxia by termination of ventilation. FIG. 4C, a representative electrocardiogram with pre-treatment of Nav1.5 polyclonal antibody and then atrial arrhythmia was induced. Atrial arrhythmia was not inducible and sinus rhythm was noted after arrhythmia induction. FIG. 4D, another representative electrocardiogram with pre-treatment of Nav1.5 polyclonal antibody and then atrial arrhythmia was induced. Atrial arrhythmia was not inducible and sinus rhythm with atrioventricular conduction block was noted after arrhythmia induction.

Atrial arrhythmia could be induced in 5 of 13 normal rats. When the rats were pre-treated with Nav1.5 polyclonal antibodies (5.12 μg), atrial arrhythmia was no longer inducible in all of 11 normal rats (5/13 vs. 0/11, P=0.035). Only sinus tachycardia was found after right atrial tachypacing and transient asphyxia (see FIG. 4C). Occasionally, atrioventricular (AV) conduction block was noted after arrhythmia induction with pre-treatment of Nav1.5 polyclonal antibody (see FIG. 4D).

FIGS. 5A and 5B show bar graphs of mean P-P intervals and R-R intervals in electrocardiogram. FIG. 5A, mean P-P intervals of control rats (CTL), those with atrial arrhythmia induction (turn off the respirator) and those with pre-treatment of Nav1.5 polyclonal antibody and atrial arrhythmia induction (turn off the respirator+Nav1.5 Ab). The mean P-P interval of atrial arrhythmia was shorter (faster rate) than that of normal sinus rhythm. The mean P-P interval of atrial arrhythmia was also shorter than that of sinus rhythm after pre-treatment of Nav1.5 polyclonal antibody and atrial arrhythmia induction. CTL means control, and Nav1.5 Ab means administration of the Nav1.5 polyclonal antibody. FIG. 5B, mean R-R intervals of control rats, those with atrial arrhythmia induction and those with pre-treatment of Nav1.5 polyclonal antibody and atrial arrhythmia induction. CTL means control, and Nav1.5 Ab means the Nav1.5 polyclonal antibody. The mean R-R interval of atrial arrhythmia was shorter (faster rate) than that of normal sinus rhythm. The mean R-R interval of atrial arrhythmia was also shorter than that of sinus rhythm after pre-treatment of Nav1.5 polyclonal antibody and atrial arrhythmia induction. From the results in FIGS. 5A and 5B, it can be seen that the mean P-P interval and R-R interval in electrocardiogram after arrhythmia induction were significantly longer in the group of antibody pre-treatment compared to that without antibody pre-treatment (statistically significant).

The result of this example confirms that the Nav1.5 polyclonal antibody of the present invention has the effect on preventing atrial arrhythmia.

In summary, the Nav1.5 polyclonal antibody of the present invention has the effect on blocking sodium ions from passing through the cardiac sodium Nav1.5 channel to treat and/or prevent cardiac arrhythmia.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A polyclonal antibody targeting sodium ion channel, which binds to a sodium Nav1.5 channel, wherein the sodium Nav1.5 channel has a Nav1.5 pore-forming region, and the polyclonal antibody targeting sodium ion channel binds to the Nav1.5 pore-forming region.

2. The polyclonal antibody targeting sodium ion channel according to claim 1, wherein the sodium Nav1.5 channel is a cardiac sodium Nav1.5 channel.

3. The polyclonal antibody targeting sodium ion channel according to claim 2, which blocks sodium ions from passing through the cardiac sodium Nav1.5 channel.

4. The polyclonal antibody targeting sodium ion channel according to claim 1, wherein the Nav1.5 pore-forming region comprises an amino acid sequence of SEQ ID NO: 1.

5. A pharmaceutical composition, comprising the polyclonal antibody targeting sodium ion channel according to claim 1, and a pharmaceutically acceptable carrier.

6. The pharmaceutical composition according to claim 5, wherein the sodium Nav1.5 channel is a cardiac sodium Nav1.5 channel.

7. The pharmaceutical composition according to claim 6, wherein the polyclonal antibody targeting sodium ion channel blocks sodium ions from passing through the cardiac sodium Nav1.5 channel.

8. The pharmaceutical composition according to claim 5, wherein the Nav1.5 pore-forming region comprises an amino acid sequence of SEQ ID NO: 1.

9. A method for treating and/or preventing cardiac arrhythmia, comprising administering to a subject in need thereof a pharmaceutical composition comprising an effective amount of the polyclonal antibody targeting sodium ion channel according to claim 1.

10. The method according to claim 9, wherein the sodium Nav1.5 channel is a cardiac sodium Nav1.5 channel.

11. The method according to claim 10, wherein the polyclonal antibody targeting sodium ion channel blocks sodium ions from passing through the cardiac sodium Nav1.5 channel.

12. The method according to claim 9, wherein the Nav1.5 pore-forming region comprises an amino acid sequence of SEQ ID NO: 1.

13. The method according to claim 9, wherein the cardiac arrhythmia is atrial arrhythmia or atrial fibrillation.

14. The method according to claim 9, wherein the pharmaceutical composition is in a dosage form for parenteral administration.

15. The method according to claim 13, wherein the arrhythmia is treated and/or prevented by blocking sodium ions from passing through the sodium Nav1.5 channel by the polyclonal antibody targeting sodium ion channel.


SEQUENCE LISTING (1 sequence)

SEQ ID NO: 1 - protein, 15 aa, synthetic construct
  REGION 1..15: note = synthesized peptide
KNYSELRHRRISDSG